(19) United States
(12) Patent Application Publication
(10) Pub. No.: US 2003/0191400 A1
(43) Pub. Date: Oct. 9, 2003

(54) System for determining values of hemodynamic parameters for a lesioned blood vessel, processor therefor, and method therefor

(75) Inventors: Evgeny Shalman, Tel Aviv (IL); Alexander Tymonkin, Tel Aviv (IL); Elhanan Dgany, Kfar Saba (IL); Orly Dgany, Kfar Saba (IL); Chen Barak, Shoham (IL); Michael Ortenburg, Kfar Yona (IL)

Correspondence Address: Harold L. Novick, Sixth Floor, 1030 Fifteenth Street, N.W., Washington, DC, 20005, US

(73) Assignee: Florence Medical Ltd., Kfar Saba (IL)

(21) Appl. No.: 10/412,335

(22) Filed: Apr. 14, 2003

Related U.S. Application Data
(63) Continuation-in-part of application No. 09/978,179, filed on Oct. 17, 2001, now Pat. No. 6,558,334.
(60) Provisional application No. 60/406,918, filed on Aug. 30, 2002.
(60) Provisional application No. 60/241,333, filed on Jan. 19, 2001.

(51) Int. Cl. A61B 5/02

(52) U.S. Cl. 600/486

(57) ABSTRACT

Quantification of the change in shape of the dicrotic notches of so-called distal pressure pulses acquired distal to a lesioned section of a lesioned blood vessel relative to the dicrotic notches of so-called proximal pressure pulses acquired proximal thereto enable determination of values of hemodynamic parameters. The envisaged hemodynamic parameters can include so-called Pulse Transmission Coefficients, non-hyperemic substitutes to the clinically accepted Fractional Flow Reserve and Coronary Flow Reserve indices, and a RC time constant indicative of the health of the vascular bed fed by a lesioned blood vessel.

### FIELD OF THE INVENTION

The invention relates to determining values of hemodynamic parameters for a lesioned blood vessel.

### BACKGROUND OF THE INVENTION

Stroke volume pumping by a left ventricle into its adjacent proximal aortic root causes the pressure of the root segment to rise and its wall to distend because it is already filled with blood, thereby creating a high pressure wave which is transmitted into the arteries. The morphology of the aortic pressure pulse corresponds to the three phases of the pressure pulse as follows: Phase I is known as the anacrotic rise occurring during early systole and correlating with the inotropic component, the gradient, and height of the anacrotic rise, and anacrotic notch being related to the rate of acceleration of blood. Phase II appears as a rounded shoulder by virtue of the continued ejection of stroke volume from the left ventricle, displacement of blood, and distension of the arterial walls which produce the rounded appearance. And Phase III appears as a descending limb due to diastolic run-off of blood. This part of the curve normally begins with a dicrotic notch as affected by blood running against the closing aortic valve separating systole from diastole. A decrease in arterial distensibility occurs with aging and in hypertension, but is most apparent in generalized arteriosclerosis. A decrease in arterial distensibility causes an increase in pulse wave velocity which in turn results in the early return of reflected waves from peripheral sites.

Early observations suggested that pressure pulse analysis is useful in evaluating the severity of atherosclerotic vascular disease. Using a classification according to the appearance of the dicrotic notch in the peripheral pressure pulse, it was demonstrated that abnormal pressure pulse with the absence of discrete dicrotic notch is associated with significant atherosclerotic vascular disease. Dawber, T. R., et al, “Characteristics of the dicrotic notch of the arterial pulse wave in coronary heart disease”, Angiology, 1973, 24(4): p. 244-55.

More recently, it was shown that abnormalities in the carotid pulse waveform with alteration or disappearance of the dicrotic notch is highly correlated with isolated aortic stenosis. O'Boyle, M. K., et al, “Duplex sonography of the carotid arteries in patients with isolated aortic stenosis: imaging findings and relation to severity of stenosis”, American Journal of Roentgenology, 1996, 166(1): p. 197-202. Cousins, A. L., et al “Prediction of aortic valvular area and gradient by noninvasive techniques”, American Heart Journal, 1978, 95(3): p. 308-15.

Furthermore, the absence of the dicrotic notch in the pulse pressure waveforn distally to aortoliac disease was almost always associated with significant proximal artery stenosis whereas its presence was found as an excellent index of normal hemodynamics. Barringer, M., et al., “The diagnosis of aortoiliac disease. A noninvasive femoral cuff technique”, Annals of Surgery, 1983, 197(2): p. 204-9.

### SUMMARY OF THE INVENTION

The present invention is based on the premise that quantification of the change in shape of dicrotic notches of so-called distal pressure pulses acquired distal to a lesioned section of a lesioned blood vessel relative to dicrotic notches of so-called proximal pressure pulses acquired proximal thereto can yield clinically important hemodynamic information regarding the lesioned section itself and/or the vascular bed fed by the lesioned blood vessel. The basic hemodynamic parameter envisaged by the present invention is a so-called Pulse Transmission Coefficient (PTC) index believed to be indicative of the cumulative effects of a lesioned section of a lesioned blood vessel and the health of the vascular bed fed thereby. PTC values can be determined based on pressure waveforms containing a series of pressure pulses acquired at rest or hyperemia as induced by the administration of a suitable vasodilatation medicament, for example, adenosine. PTC values can be determined for both single lesioned and multi-lesioned blood vessels. Four different techniques for determining PTC values for a lesioned blood vessel are described in detail hereinbelow Whilst PTC values are believed to have clinical significance in their own right, non-hyperemic PTC values together with Base Pressure Gradients (BPGs) are acquired at rest by definition, can be employed for calculating non-hyperemic substitutes to the clinically accepted Fractional Flow Reserve (FFR) and Coronary Flow Reserve (CFR) indices with similar cutoff values, namely, <0.75 and <2, respectively, being indicative of the need for intervention. Within the context of the present invention, the non-hyperemic FFR substitute is termed Lesion Severity Index (LSI). LSI values can be determined for the single lesion of a single lesioned blood vessel or for each lesion of a multi-lesioned blood vessel.

Additionally, inasmuch that a vascular bed's compliance C and resistivity R can be regarded as being respectively equivalent to capacitance C and resistance R, a vascular bed can be considered analogous to a parallel RC circuit whereby a PTC value for a lesioned blood vessel can be determined to yield a RC time constant indicative of the health of the vascular bed fed thereby

### BRIEF DESCRIPTION OF THE DRAWINGS

In order to understand the invention and to see how it can be carried out in practice, preferred embodiments will now be described, by way of non-limiting examples only, with reference to the accompanying drawings in which:

FIG. 1 is a block diagram of a system for determining values of hemodynamic parameters for a lesioned blood vessel in accordance with the present invention;

FIG. 2 is a graph showing an exemplary proximal pressure waveform acquired proximal to a lesioned section of a lesioned blood vessel;

FIG. 3 is a graph showing an exemplary distal pressure waveform acquired distal to a non-severely lesioned section of a lesioned blood vessel;

FIG. 4 is a graph showing an exemplary distal pressure waveform acquired distal to a severely lesioned section of a lesioned blood vessel;

FIG. 5 showing an exemplary measured pressure pulse P(t) and its low pass filtered derivative Plow(t);

FIG. 6 is a graph showing the measured pressure pulse P(t) of FIG. 5 and the function dP(t) where dP(t)=P(t)−Plow(t) for determining the value of a PTC(E) index in accordance with the present invention;

FIG. 7 is a pictorial representation showing the area of a dicrotic notch for determining the value of a PTC(A) index in accordance with the present invention;

FIG. 8 is a pictorial representation showing the approximation of the area of the dicrotic notch of FIG. 7 to that of a scalene triangle;

FIG. 9A is a graphical representation showing the area of the leading portion of a distal pressure pulse relative to its entire area for use in determining the value of a PTC(B) index in accordance with the present invention;

FIG. 9B is a graphical representation showing the area of the leading portion of a proximal pressure pulse relative to its entire area for use in determining the value of a PTC(B) index in accordance with the present invention;

FIG. 10A is a graphical representation showing the height of the dicrotic notch of a distal pressure pulse relative to its maximum height for use in determining the value of a PTC(H) index in accordance with the present invention;

FIG. 10B is a graphical representation showing the height of the dicrotic notch of a proximal pressure pulse relative to its maximum height for use in determining the value of a PTC(H) index in accordance with the present invention;

FIG. 11 is a graph plotting LSI values against FFR values for a clinical study of 92 human patients; and

FIG. 12 is a graph plotting non-hyperemic CFR values against true CFR values for a clinical study of 29 human patients.

### DESCRIPTION OF PREFERRED EMBODIMENTS

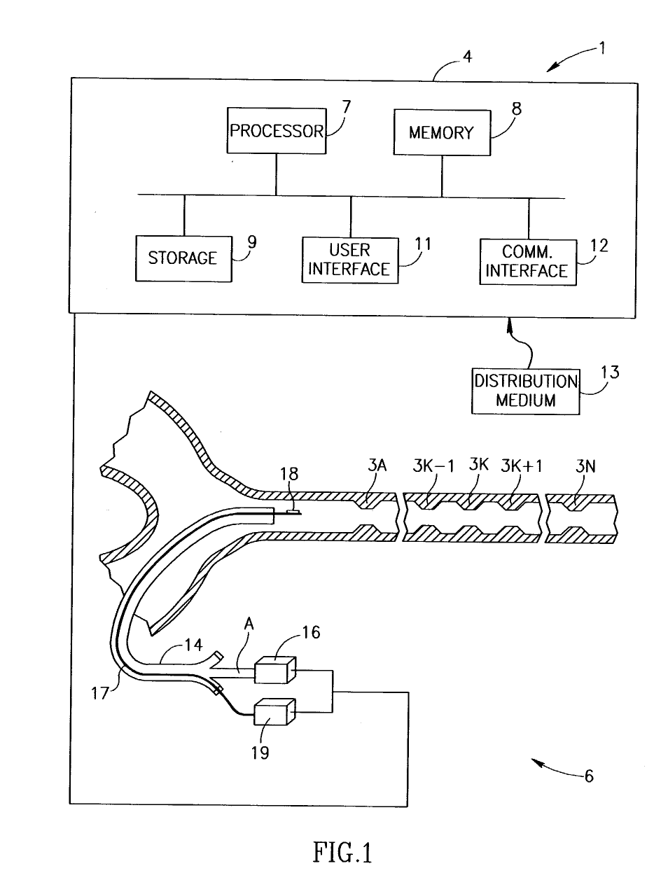

FIG. 1 shows a system 1 for determining values of hemodynamic parameters for a lesioned blood vessel 2 having one or more lesions 3. The system 1 includes a general purpose digital computer 4 and intravascular pressure measurement apparatus 6 for acquiring pressure measurements at different locations along the blood vessel 2. The pressure measurements are typically acquired in the form of a pressure waveform of a series of consecutive pressure pulses. The computer 4 includes a processor 7 programmed to determine values of hemodynamic parameters for the blood vessel 2, system memory 8, nonvolatile storage 9, a user interface 11, and a communication interface 12. The constitution of each of these elements is well known and each performs its conventional function as known in the art and accordingly will not be described in greater detail. In particular, the system memory 8 and the non-volatile storage 9 are employed to store a working copy and a permanent copy of the programming instructions implementing the present invention. The permanent copy of the programming instructions to practice the present invention may be loaded into the non-volatile storage 9 in the factory, or in the field, through communication interface 12, or through distribution medium 13. Any one of a number of recordable medium such as tapes, CD-ROM, DVD and so forth may be employed to store the programming instructions for distribution purposes.

The intravascular pressure measurement apparatus 6 includes a guiding catheter 14 connected to a fluid filled pressure transducer 16 deployed outside of a patient's body at position A for continuously acquiring aortic pressure for use as a baseline for correcting pressure measurements to compensate for various factors, for example, physiological changes in pressure, breathing, patient movement, and the like, which may influence intravascular pressure measurements since they are not acquired simultaneously. An exemplary guiding catheter 14 is the Ascent JL4 catheter commercially available from Medtronic, USA whilst an exemplary fluid filled pressure transducer 16 is commercially available from Biometrix, Jerusalem, Israel. The intravascular pressure measurement apparatus 6 also includes a pressure guide wire 17 with a pressure transducer 18 at its tip for acquiring pressure measurements along the blood vessel. The pressure transducer 18 is connected to a signal conditioning device 19. An exemplary pressure guide wire 17 is the PressureWire® pressure guide wire commercially available from Radi Medical Systems, Uppsala, Sweden whilst an exemplary signal conditioning device 19 is also commercially available from Radi Medical Systems.

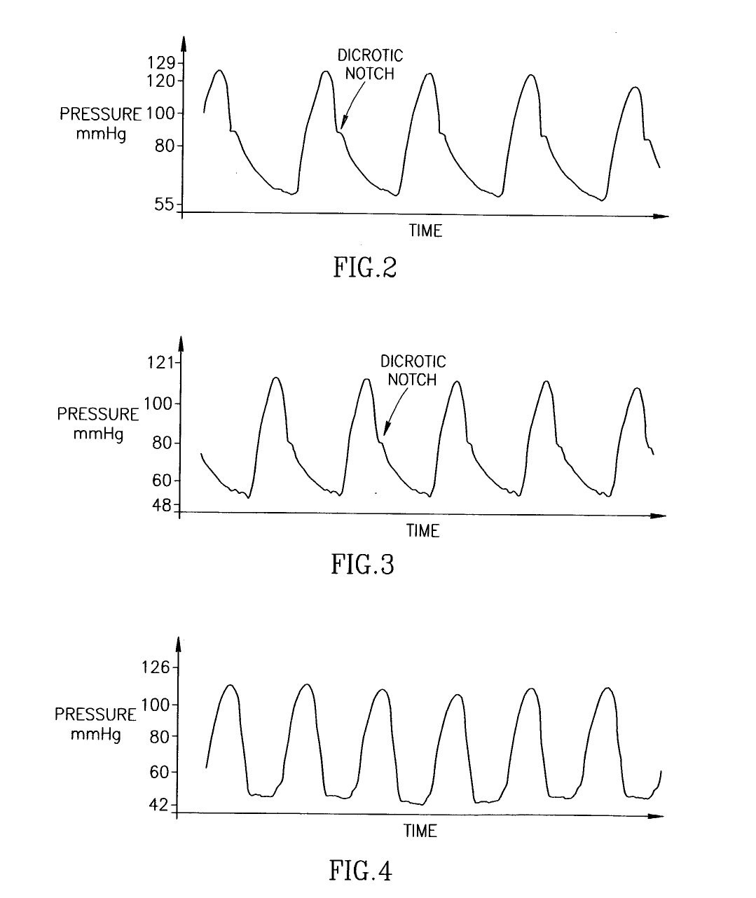

FIG. 2 depicts an exemplary proximal rest pressure waveform acquired proximal to a lesioned section of a lesioned blood vessel, the pressure waveform including a series of consecutive pressure pulses each having a dicrotic notch which typically continues in the distal rest pressure waveform in the case of a non-severely lesioned blood vessel (see FIG. 3) but discontinues in the case of a severely lesioned blood vessel (see FIG. 4).

Determination of PTC(E) Index

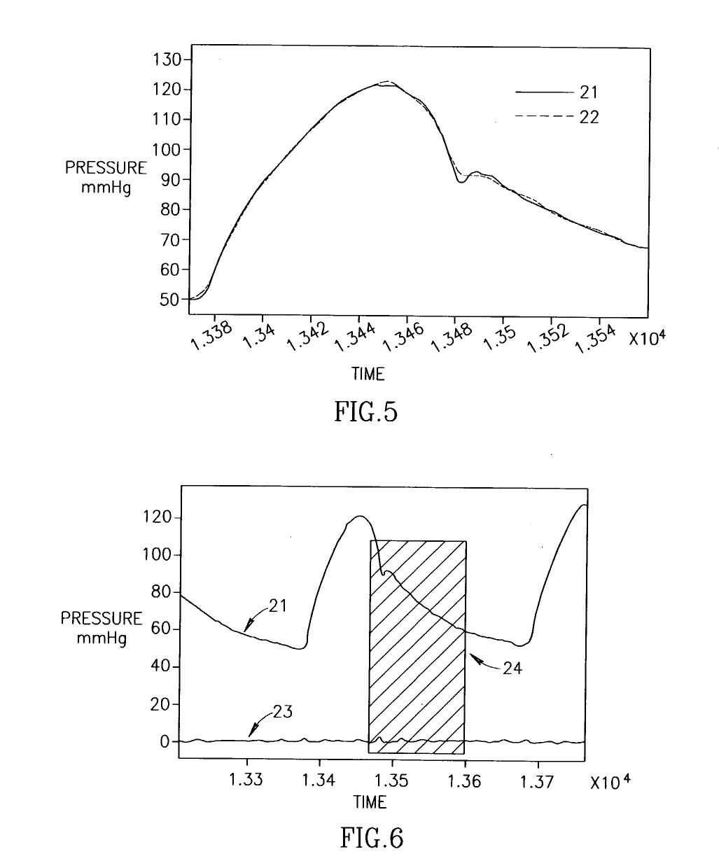

The present invention proposes a first hemodynamic parameter PTC(E) quantifying a change in the shape of the dicrotic notch of a distal pressure pulse with respect to the dicrotic notch of a proximal pressure pulse in accordance with the relationship: PTC(E) α Edistal/Eproximal where Edistal is the energy of the high frequency component of the dicrotic notch of a distal pressure pulse and Eproximal is the energy of the high frequency component of the dicrotic notch of a proximal pressure pulse. The energy of the high frequency component of the dicrotic notch of a pressure pulse is given by the standard deviation of dP(t) where dP(t)=P(t)−Plow(t), P(t) being a measured pressure pulse and Plow(t) its low pass filtered derivative containing, say, the first 6 harmonics of the measured pressure pulse P(t). FIG. 5 shows a graph with a measured pressure pulse P(t) 21 (full line) and its low pass filtered derivative Plow(t) 22 (dotted line). FIG. 6 shows the differential pressure pulse dP(t) 23 and an exemplary Region Of Interest (ROI) 24 for determining the energy of the high frequency component of a dicrotic notch. Other high frequency components of the differential pressure pulse dP(t) can be observed at the occurrences of maximum pressure and minimum pressure. The ROIs for determining Edistal and Eproximal may be invoked manually or automatically using zeroes of the function dP(t) before determining the value of the PCT(E) index. A Plow(t) low pass filtered derivative can contain more or less harmonics of the measured pressure pulse P(t), say, between five to seven.

Determination of PTC(A) Index

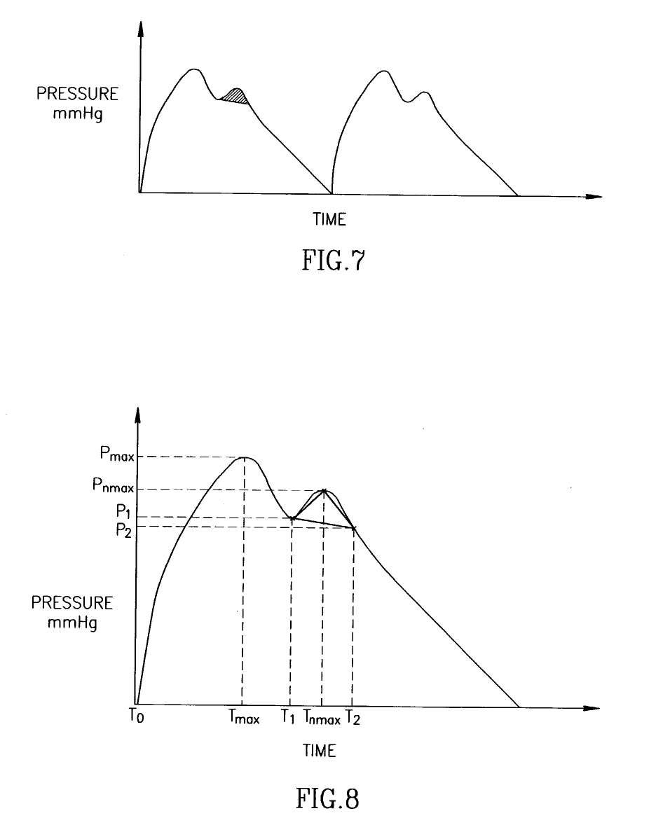

The present invention proposes a second hemodynamic parameter PTC(A) quantifying a change in the shape of the dicrotic notch of a distal pressure pulse with respect to the dicrotic notch of a proximal pressure pulse in accordance with the relationship: PTC(A) α Adistal/Aproximal where Adistal is the area of the dicrotic notch of a distal pressure pulse and Aproximal is the area of the dicrotic notch of a proximal pressure pulse (see FIG. 7). For computational ease, the shaded area of the dicrotic notch of a pressure pulse is approximated as that of a scalene triangle having vertices which lie thereon. The coordinates of the vertices are as follows: (T1,P1) where T1 corresponds to the occurrence of the first local post systolic minimum of the pressure pulse distinguishable by a sign change in the 1^{st} order differential dP/dt; (Tnmax,Pnmax) corresponds to the occurrence of the local maximum pressure of the dicrotic notch; and (T2,P2) where T2=T1+(Tmax−T0)/3 where Tmax corresponds to the occurrence of maximum pressure Pmax of the pressure pulse, and T0 corresponds to the occurrence of minimum pressure (see FIG. 8).

Determination of PTC(B) Index

The present invention proposes a third hemodynamic parameter PTC(B) quantifying a change in the shape of the leading portion of a distal pressure pulse with respect to the leading portion of a proximal pressure pulse in accordance with the relationship:

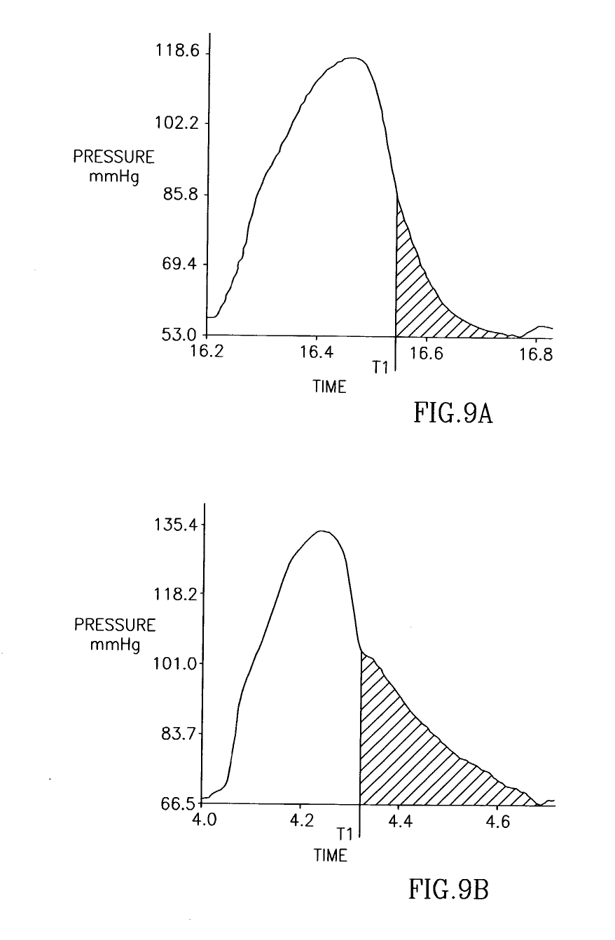

PTC(B)α (Adistalnotch/Adistalpulse)/(Aproximalnotch/Aproximalpulse)

where Adistalnotch is the shaded area under the leading portion of a distal pressure pulse, and Adistalpulse is its entire area (see FIG. 9A); and Aproximalnotch is the shaded area under the leading portion of a proximal pressure pulse, and Aproximalpulse is its entire area (see FIG. 9B). The leading portion of a pressure pulse is preferably defined as being prior to the occurrence of its first local post systolic minimum denoted T1.

Determination of PTC(H) Index

The present invention proposes a fourth hemodynamic parameter PTC(H) quantifying a change in the shape of the dicrotic notch of a distal pressure pulse with respect to the dicrotic notch of a proximal pressure pulse in accordance with the relationship:

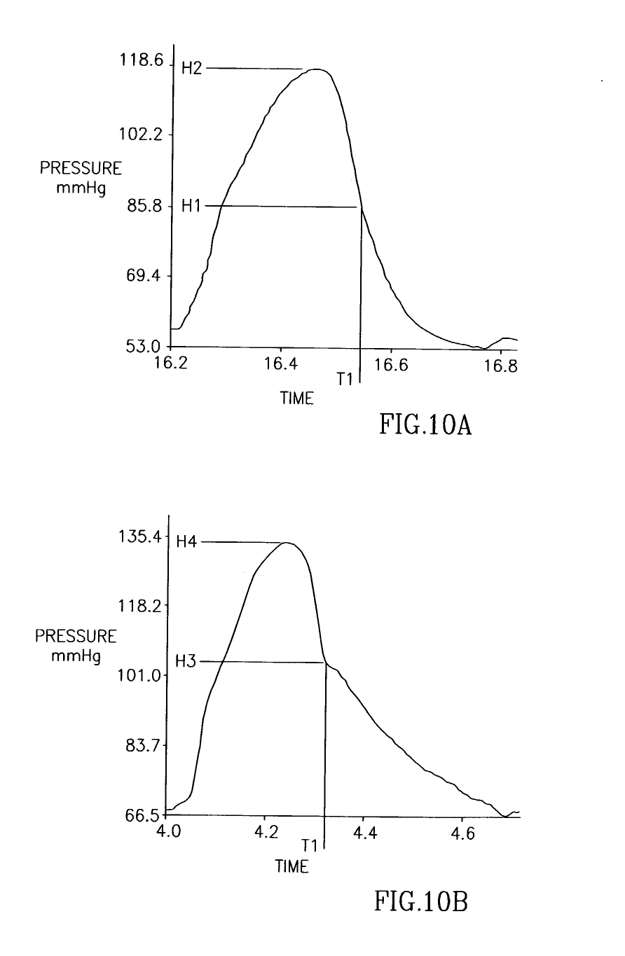

PTC(H)α (Hdistalnotch/Hdistalpulse)/(Hproximalnotch/Hproximalpulse)

where Hdistalnotch is the height H1 of the dicrotic notch of a distal pressure pulse, and Hdistalpulse is its maximum height H2 (see FIG. 10A); and Hproximalnotch is the height H3 of the dicrotic notch of a proximal pressure pulse, and Hproximalpulse is its maximum height H4 (see FIG. 10B). The height of the dicrotic notch of a pressure pulse is preferably determined at the occurrence of its first local post systolic minimum denoted T1.

Determination of Lesion Severity Index (LSI)

The non-hyperemic PTC value for a lesioned blood vessel may be employed together with the Base Pressure Gradient (BPG) therefor for arriving at a non-hyperemic FFR substitute with a similar cutoff value <0.75 indicative of the need for intervention. Thus, LSI is a function of non-hyperemic PTC and BPG values in general and, in greater particularity, is a function of a quadratic equation of the form: LSI α (a+bK_{LBP}+cK_{LBP}²) where K_{LBP} α (log PTC)/BPG, and a, b and c are coefficients. In practice, in the case of small PTC values <0.3, it has been found that LSI values more accurately correlate to actual FFR values by adding another term to the above LSI equation as follows:

LSI α (a+bK_{LBP}+cK_{LBP}²)(d+eK_{LBP}) where d and e are also coefficients.

The BPG is preferably a corrected value in accordance with the relationship:

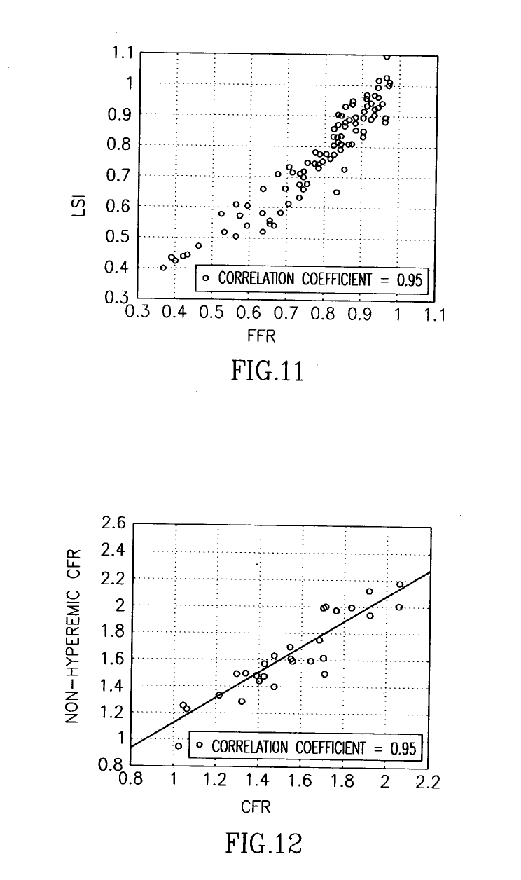

BPG α BPG_{diastolicmax}/P_{aortic} where BPG_{diastolicmax} is the measured BPG value acquired at maximum diastole and P_{aortic} is the aortic pressure. FIG. 11 shows that the LSI values for a clinical study of 92 human patients have a high correlation with true FFR values.

Determination of Individual LSI Values for the Lesions of a Multi-Lesioned Blood Vessel

The non-hyperemic PTC value for a multi-lesioned blood vessel may be employed together with the individual Base Pressure Gradient (BPG) across each of its lesions for arriving at non-hyperemic LSI substitutes to the individual FFR values obtainable as illustrated and described in commonly assigned PCT International Application PCT/IL02/00694 published under WO03/022122 incorporated herein by reference. Mathematically speaking, the k^{th} lesion of a multi-lesioned blood vessel is given by the relationship:

LSIₖα (log PTC)/BPGₖ

where the PTC value is acquired across the entire lesioned section of the multi-lesioned blood vessel, and BPGₖ is acquired across the k^{th} lesion.

Determination of Non-Hyperemic Coronary Flow Reserve (CFR)

Similarly, the non-hyperemic PTC value for a lesioned blood vessel may be employed together with the Base Pressure Gradient (BPG) therefor for arriving at a non-hyperemic substitute for CFR with a similar cutoff value <2 indicative of the need for intervention. In accordance with the relationship CFR α \sqrt{HPG/BPG} where HPG is the hyperemic pressure gradient and BPG is the base pressure gradient across the lesioned section of a lesioned blood vessel as set out in commonly assigned U.S. Pat. No. 6,471,656, the contents of which are incorporated by reference, a non-hyperemic CFR value can be yielded for a lesioned blood vessel in accordance with the relationship: CFR≈\sqrt{P_{α}(1−LSI)}/\sqrt{BRG}. FIG. 12 shows that the non-hyperemic CFR values for a clinical study of 29 human patients have a high correlation with true CFR values.

Determination of RC Time Constant

The non-hyperemic PTC value for a lesioned blood vessel may be employed together with the BPG therefor for arriving at a RC time constant characterizing the vascular bed fed thereby. Thus, a RC time constant is a function of non-hyperemic PTC and BPG values in general and is preferably determined in accordance with a linear equation of the form:

RC time constant α (a K_{LBP}+b) where K_{LBP}=(log PTC)/BPG as before, a and b are coefficients.

While the invention has been described with respect to a limited number of embodiments, it will be appreciated that many variations, modifications, and other applications of the invention can be made within the scope of the appended claims. For example, without intending to limit the scope of the appended claims, they recite that a PTC value for a lesioned blood vessel is determined from a single dicrotic pressure pulse and a single proximal pressure pulse. In point of fact, these pressure pulses are preferably the median pressure pulses respectively of a series of distal pressure pulses, and a series of proximal pressure pulses but equally may be averaged pressure pulses, and the like. The scope of the appended claims is also intended to encompass alternative techniques for determining a PTC value for a lesioned blood vessel, for example, the average or median PTC value of a series of PTC values each calculated from a single dicrotic pressure pulse and a single proximal pressure pulse, and the like.

1. A system for determining the values of hemodynamic parameters for a lesioned blood vessel, the system comprising:
(a) intravascular pressure measurement apparatus for acquiring pressure measurements in a blood vessel during continuous blood flow therethrough; and
(b) a processor for determining the value of at least one hemodynamic parameter based on the change in shape of the dicrotic notches of one or more distal pressure pulses acquired distal to a lesioned section of a lesioned blood vessel with respect to the dicrotic notches of one or more proximal pressure pulses acquired proximal to the lesioned section of the lesioned blood vessel.

2. The system according to claim I wherein the hemodynamic parameter is a Pulse Transmission Coefficient index PTC(E) where
PTC(E) α Edistal/Eproximal
where Edistal is the energy of the high frequency component of the dicrotic notch of a distal pressure pulse and Eproximal is the energy of the high frequency component of the dicrotic notch of a proximal pressure pulse.

3. The system according to claim 2 wherein the energy of the high frequency component of a dicrotic notch is given by the standard deviation of dP(t) where dP(t)=P(t)−Plow(t), P(t) being a measured pressure pulse and Plow(t) its low pass filtered derivative.

4. The system according to claim 1 wherein the hemodynamic parameter is a Pulse Transmission Coefficient index PTC(A) where
PTC(A) α Adistal/Aproximal
where Adistal is the area of the dicrotic notch of a distal pressure pulse and Aproximal is the area of the dicrotic notch of a proximal pressure pulse.

5. The system according to claim 4 wherein the area of the dicrotic notch of a pressure pulse is approximated as the area of a triangle whose vertices lie thereon.

6. The system according to claim 5 wherein the vertices of the triangle are as follows: (T1,P1) where T1 corresponds to the occurrence of the first local post systolic minimum of the pressure pulse; (Tnmax,Pnmax) corresponds to the occurrence of the local maximum pressure of the dicrotic notch; and (T2,P2) where T2=T1+(Tmax−T0)/3 where Tmax corresponds to the occurrence of maximum pressure Pmax of the pressure pulse, and T0 corresponds to the occurrence of minimum pressure.

7. The system according to claim 1 wherein the hemodynamic parameter is a Pulse Transmission Coefficient index PCT(B) where
PTC(B) α (Adistalnotch/Adistalpulse)/(Aproximalnotch/Aproximalpulse)
where Adistalnotch is the area under the leading portion of a distal pressure pulse, and Adistalpulse is its entire area; and Aproximalnotch is the area under the leading portion of a proximal pressure pulse, and Aproximalpulse is its entire area.

8. The system according to claim 7 wherein the leading portion of a pressure pulse is defined as being prior to the occurrence of its first local post systolic minimum.

9. The system according to claim 1 wherein the hemodynamic parameter is a Pulse Transmission Coefficient index PCT(H) where
PTC(H) α (Hdistalnotch/Hdistalpulse)/(Hproximalnotch/Hproximalpulse)
where Hdistalnotch is the height of the dicrotic notch of a distal pressure pulse, Hdistalpulse is its maximum height, Hproximalnotch is the height of the dicrotic notch of a proximal pressure pulse, and Hproximalpulse is its maximum height.

10. The system according to claim 9 wherein the height of the dicrotic notch of a pressure pulse is determined at its first local post systolic minimum.

11. The system according to claim 1 wherein the hemodynamic parameter is a non-hyperemic substitute Lesion Severity Index (LSI) for the Fractional Flow Reserve (FFR) index for a lesioned blood vessel, LSI being a function of non-hyperemic PTC and BPG values, and having a <0.75 cutoff value indicative of the need for intervention.

12. The system according to claim 11 wherein LSI α (a+bK_{LBP}+cK_{LBP}²) where K_{LBP}α (log PTC)/BPG, and a, b and c are coefficients.

13. The system according to claim 12 wherein for PTC<0.3:
LSI α (a+bK_{LBP}+cK_{LBP}²)(d+eK_{LBP}) where d and e are also coefficients.

14. The system according to claim 12 wherein BPG α BPG_{diastolicmax}/P_{aortic} where BPG_{diastolicmax} is the measured BPG value acquired at maximum diastole and P_{aortic} is the aortic pressure.

15. The system according to claim 1 wherein the hemodynamic parameter is a non-hyperemic substitute Lesion Severity Index (LSIₖ) for the individual Fractional Flow Reserve (FFR) index for a k^{th} lesion of a multi-lesioned blood vessel in accordance with the relationship LSIₖ α (log PTC)/BPGₖ where PTC is acquired across the entire lesioned section of the multi-lesioned blood vessel, and BPGₖ is acquired across its k^{th} lesion.

16. The system according to claim 1 wherein the hemodynamic parameter is a non-hyperemic substitute for the Coronary Flow Reserve (CFR) index for a lesioned blood vessel, the non-hyperemic CFR value being a function of non-hyperemic PTC and BPG values determined therefrom, and having a <2 cutoff value indicative of the need for intervention.

17. The system according to claim 1 wherein the hemodynamic parameter is a RC time constant for the vascular bed fed by the lesioned blood vessel being a function of non-hyperemic PTC and BPG values determined therefrom.

18. The system according to claim 15 wherein
RC time constant α (a K_{LBP}+b)
where K_{LBP}=(log PTC)/BPG, and a and b are constants.

19. For use with intravascular pressure measurement apparatus capable of acquiring pressure measurements in a blood vessel during continuous blood flow therethrough, a processor capable of executing the following steps:
(a) processing information relating to the shape of the dicrotic notches of one or more proximal pressure pulses acquired proximal to a lesioned section of a lesioned blood vessel;
(b) processing information relating to the shape of the dicrotic notches of one or more distal pressure pulses acquired distal to the lesioned section of the lesioned blood vessel; and
(c) determining the value of at least one hemodynamic parameter based on the change in shape of the dicrotic notches of one or more distal pressure pulses acquired distal to a lesioned section of a lesioned blood vessel with respect to the dicrotic notches of one or more proximal pressure pulses acquired proximal to the lesioned section of the lesioned blood vessel.

20. The processor according to claim 19 wherein the hemodynamic parameter is a Pulse Transmission Coefficient index PTC(E) where
PTC(E) α Edistal/Eproximal
where Edistal is the energy of the high frequency component of the dicrotic notch of a distal pressure pulse, and Eproximal is the energy of the high frequency component of the dicrotic notch of a proximal pressure pulse.

21. The processor according to claim 20 wherein the energy of the high frequency component of a dicrotic notch is given by the standard deviation of dP(t) where dP(t)=P(t)−Plow(t), P(t) being a measured pressure pulse and Plow(t) its low pass filtered derivative.

22. The processor according to claim 19 wherein the hemodynamic parameter is a Pulse Transmission Coefficient index PTC(A) where
PTC(A) α Adistal/Aproximal
where Adistal is the area of the dicrotic notch of a distal pressure pulse, and Aproximal is the area of the dicrotic notch of a proximal pressure pulse.

23. The processor according to claim 22 wherein the area of the dicrotic notch of a pressure pulse is approximated as the area of a triangle whose vertices lie thereon.

24. The processor according to claim 23 wherein the vertices of the triangle are as follows: (T1,P1) where T1 corresponds to the occurrence of the first local post systolic minimum of the pressure pulse; (Tnmax,Pnmax) corresponds to the occurrence of the local maximum pressure of the dicrotic notch; and (T2,P2) where T2=T1+(Tmax−T0)/3 where Tmax corresponds to the occurrence of maximum pressure Pmax of the pressure pulse, and T0 corresponds to the occurrence of minimum pressure.

25. The processor according to claim 19 wherein the hemodynamic parameter is a Pulse Transmission Coefficient index PCT(B) where
PTC(B) α (Adistalnotch/Adistalpulse)/(Aproximalnotch/Aproximalpulse)
where Adistalnotch is the area under the leading portion of a distal pressure pulse, and Adistalpulse is its entire area; and Aproximalnotch is the area under the leading portion of a proximal pressure pulse, and Aproximalpulse is its entire area.

26. The processor according to claim 25 wherein the leading portion of a pressure pulse is defined as being prior to the occurrence of its first local post systolic minimum.

27. The processor according to claim 19 wherein the hemodynamic parameter is a Pulse Transmission Coefficient index PCT(H) where
PTC(H) α (Hdistalnotch/Hdistalpulse)/(Hproximalnotch/Hproximalpulse)
where Hdistalnotch is the height of the dicrotic notch of a distal pressure pulse, Hdistalpulse is its maximum height Hproximalnotch is the height of the dicrotic notch of a proximal pressure pulse, and Hproximalpulse is its maximum height.

28. The processor according to claim 27 wherein the height of the dicrotic notch of a pressure pulse is determined at its first local post systolic minimum.

29. The processor according to claim 19 wherein the hemodynamic parameter is a non-hyperemic substitute Lesion Severity Index (LSI) for the Fractional Flow Reserve (FFR) index for a lesioned blood vessel, LSI being a function of non-hyperemic PTC and BPG values, and having a <0.75 cutoff value indicative of the need for intervention.

30. The processor according to claim 29 wherein LSI α (a+bK_{LBP}+cK_{LBP}²) where K_{LBP} α (log PTC)/BPG, and a, b and c are coefficients.

31. The processor according to claim 30 wherein for PTC<0.3:
LSI α (a+bK_{LBP}+cK_{LBP}²)(d+eK_{LBP}) where d and e are also coefficients.

32. The processor according to claim 30 wherein BPG α BPG_{diastolicmax}/P_{aortic} where BPG_{diastolicmax} is the measured BPG value acquired at maximum diastole and P_{aortic} is the aortic pressure.

33. The processor according to claim 19 wherein the hemodynamic parameter is a non-hyperemic substitute Lesion Severity Index (LSIₖ) for the individual Fractional Flow Reserve (FFR) index for a k^{th} lesion of a multi-lesioned blood vessel in accordance with the relationship LSIₖ α (log PTC)/BPGₖ where PTC is acquired across the entire lesioned section of the multi-lesioned blood vessel, and BPGₖ is acquired across its k^{th} lesion.

34. The processor according to claim 19 wherein the hemodynamic parameter is a non-hyperemic substitute for the Coronary Flow Reserve (CFR) index for a lesioned blood vessel, the non-hyperemic CFR value being a function of non-hyperemic PTC and BPG values determined therefrom, and having a <2 cutoff value indicative of the need for intervention.

35. The processor according to claim 19 wherein the hemodynamic parameter is a RC time constant for the vascular bed fed by the lesioned blood vessel as a function of non-hyperemic PTC and BPG values determined therefrom.

36. The processor according to claim 35 wherein
RC time constant α (a K_{LBP}+b)
where K_{LBP}=(log PTC)/BPG, and a and b are constants.

37. A method for determining the values of hemodynamic parameters for a lesioned blood vessel, the method comprising the steps of
(a) deploying an intravascular pressure measurement apparatus for acquiring pressure measurements in a blood vessel during continuous blood flow therethrough; and
(b) determining the value of at least one hemodynamic parameter based on the change in shape of the dicrotic notches of one or more distal pressure pulses acquired distal to a lesioned section of a lesioned blood vessel with respect to the dicrotic notches of one or more proximal pressure pulses acquired proximal to the lesioned section of the lesioned blood vessel.

38. The method according to claim 37 wherein the hemodynamic dynamic is a Pulse Transmission Coefficient index PTC(E) where
PTC(E) α Edistal/Eproximal
where Edistal is the energy of the high frequency component of the dicrotic notch of a distal pressure pulse, and Eproximal is the energy of the high frequency component of the dicrotic notch of a proximal pressure pulse.

39. The method according to claim 38 wherein the energy of the high frequency component of the dicrotic notch of a pressure pulse is given by the standard deviation of dP(t) where dP(t)=P(t)−Plow(t), P(t) being the measured pressure pulse and Plow(t) its low pass filtered derivative.

40. The method according to claim 37 wherein the hemodynamic parameter is a Pulse Transmission Coefficient index PTC(A) where
PTC(A) α Adistal/Aproximal
where Adistal is the area of the dicrotic notch of a distal pressure pulse, and Aproximal is the area of the dicrotic notch of a proximal pressure pulse.

41. The method according to claim 40 wherein the area of the dicrotic notch of a pressure pulse is approximated as the area of a triangle whose vertices lie thereon.

42. The method according to claim 41 wherein the vertices of the triangle are as follows: (T1,P1) where T1 corresponds to the occurrence of the first local post systolic minimum of the pressure pulse; (Tnmax,Pnmax) corresponds to the occurrence of the local maximum pressure of the dicrotic notch; and (T2,P2) where T2=T1+(Tmax−T0)/3 where Tmax corresponds to the occurrence of maximum pressure Pmax of the pressure pulse, and T0 corresponds to the occurrence of minimum pressure.

43. The method according to claim 37 wherein the hemodynamic parameter is a Pulse Transmission Coefficient index PCT(B) where
PTC(B) α (Adistalnotch/Adistalpulse)/(Aproximalnotch/Aproximalpulse)
where Adistalnotch is the area under the leading portion of a distal pressure pulse, and Adistalpulse is its entire area; and Aproximalnotch is the area under the leading portion of a proximal pressure pulse, and Aproximalpulse is its entire area.

44. The method according to claim 43 wherein the leading portion of a pressure pulse is defined as being prior to the occurrence of its first local post systolic minimum.

45. The method according to claim 37 wherein the hemodynamic parameter is a Pulse Transmission Coefficient index PCT(H) where
PTC(H) α (Hdistalnotch/Hdistalpulse)/(Hproximalnotch/Hproximalpulse)
where Hdistalnotch is the height of the dicrotic notch of a distal pressure pulse, Hdistalpulse is its maximum height, Hproximalnotch is the height of the dicrotic notch of a proximal pressure pulse, and Hproximalpulse is its maximum height.

46. The method according to claim 45 wherein the height of the dicrotic notch of a pressure pulse is determined at its first local post systolic minimum.

47. The method according to claim 37 wherein the hemodynamic parameter is a non-hyperemic substitute Lesion Severity Index (LSI) for the Fractional Flow Reserve (FFR) index for a lesioned blood vessel, LSI being a function of non-hyperemic PTC and BPG values, and having a <0.75 cutoff value indicative of the need for intervention.

48. The method according to claim 47 wherein LSI α (a+bK_{LBP}+cK_{LBP}²) where K_{LBP} α (log PTC)/BPG, and a, b and c are coefficients.

49. The method according to claim 48 wherein for PTC<0.3:
LSI α (a+bK_{LBP}+cK_{LBP}²)(d+eK_{LBP}) where d and e are also coefficients.

50. The method according to claim 48 wherein BPG α BPG_{diastolicmax}/P_{aortic} where BPG_{diastolicmax} is the measured BPG value acquired at maximum diastole and P_{aortic} is the aortic pressure.

51. The method according to claim 37 wherein the hemodynamic parameter is a non-hyperemic substitute Lesion Severity Index (LSIₖ) for the individual Fractional Flow Reserve (FFR) index for a k^{th} lesion of a multi-lesioned blood vessel in accordance with the relationship LSIₖ=(log PTC)/BPGₖ where PTC is acquired across the entire lesioned section of the multi-lesioned blood vessel, and BPGₖ is acquired across its k^{th} lesion.

52. The method according to claim 37 wherein the hemodynamic parameter is a non-hyperemic substitute for the Coronary Flow Reserve (CFR) index for a lesioned blood vessel, the non-hyperemic CFR value being a function of non-hyperemic PTC and BPG values determined therefrom, and having a <2 cutoff value indicative of the need for intervention.

53. The method according to claim 37 wherein the hemodynamic parameter is a RC time constant for the vascular bed fed by the lesioned blood vessel as a function of non-hyperemic PTC and BPG values determined therefrom.

54. The method according to claim 53 wherein
RC time constant α (a K_{LBP}+b)
where K_{LBP}=(log PTC)/BPG, and a and b are constants.
